(19) United States
(12) Patent Application Publication
(10) Pub. No.: US 2022/0020504 A1
(43) Pub. Date: Jan. 20, 2022

(54) SYSTEM AND METHOD FOR VERIFIED REPORTING OF ILLNESS STATES USING DISPARATE DATASETS

(71) Applicant: Johnson & Johnson Consumer Inc., Skillman, NJ (US)

(72) Inventors: Sumeet Narula, Chalfont, PA (US); Matt Weight, Highland, UT (US); Gavin McDonough, Sedona, AZ (US); Christopher Jones, Middlesex (GB); Derek Hood, Minneapolis, MN (US); Carina Nicklaw, San Diego, CA (US); Kristopher Kohl, San Diego, CA (US)

(21) Appl. No.: 17/488,022

(22) Filed: Sep. 28, 2021

Related U.S. Application Data
(62) Division of application No. 15/150,070, filed on May 9, 2016, now abandoned.
(60) Provisional application No. 62/158,821, filed on May 8, 2015.

(51) Int. Cl. G16H 50/80; G06Q 30/02; G16H 50/70; G06F 16/29; G16Z 99/00

(52) U.S. Cl. CPC G16H 50/80; G06Q 30/0269; G16Z 99/00; G06F 16/29; G16H 50/70

(57) ABSTRACT

Illness states are reported to create disparate data sets associated with illness reports. The illness reports are verified by comparing and correlating information in the data sets. Illness reports are indicated by location-specific markers displayed on a map of a particular geographical region of interest by a computing device having a display screen. Illness reports having associated data in both data sets are considered to be verified, and thus to have a higher level of relevance as more reliable, more persuasive, more severe, and/or more accurate. Verification indicators are displayed on the display screen in association with each marker for which the associated illness report has been verified. Accordingly, reported illnesses are displayed on a geographically-relevant basis in differentiated fashion to provide a visual indication of verified and unverified reported illnesses.

This application claims the benefit of priority, under 35 U.S.C. § 119(e), of U.S. Provisional Patent Application No. 62/158,821, filed May 8, 2015, the entire disclosure of which is hereby incorporated herein by reference.

### FIELD OF THE INVENTION

### Field of the Invention

The present invention relates generally to public health, and more particularly to a system and method for verified reporting of illness states.

### Discussion of Related Art

Occurrences of illnesses are reported in a variety of ways, and are tracked for by various governmental entities, as well as by private entities. By way of example, illness are reported by individuals when visiting a healthcare provider. Accordingly, illness reports including associated information are reflected in corresponding medical records and/or associated insurance claims records (collectively referred to herein as “healthcare records”). Healthcare provider data from such records is aggregated and reviewed, so that illness can be tracked for various purposes, including for epidemiological purposes. Accordingly, illness data has been associated, for tracking purposes, with geographical areas or regions.

Data feeds reflecting data aggregated from healthcare records are commercially available in the marketplace. For example, IMS Health Incorporated of Danbury, Conn. provides commercially available data feeds reflecting data aggregated from healthcare/electronic medical record and/or insurance claim records. The data in such data feeds reflect the granularity of the underlying structure of the data as it is reported. For example, if the data is gathered from insurance claim records, the data will be matched to a standardized index/code/naming/claim structure used to process such claims. For example, the underlying data may reflect a reported “influenza-like illness (ILI)”, because that there is an associated code/option to the healthcare provider for selecting this reported condition.

Further, the underlying data reflects the granularity resulting from the aggregation process. Accordingly, each reported illness may be reported by an individual having a specific home address, but aggregation of the data may be done on a state, county, zip code, township, or other regional market basis. Accordingly, the data available in the data feed reflects illness data for geographical market regions generally. For example, a commercially-available data feed from IMS Health includes a data record including XML-formatted content identifying a geographical location such as a market region (e.g., Philadelphia metropolitan area), and a reported illness event type (e.g., cold, flu, or allergy). Further, the record may include a severity value, which may reflect a calculated or otherwise-determined reference point on a severity scale representing degrees of severity (e.g., on a scale of 0-12, with 12 indicating a highest severity).

Such data feeds are somewhat limited in nature, as they fail to capture illness states that are not reported to healthcare providers, or for which there is no corresponding healthcare record. Further, they capture illness states for purposes of medical/insurance claims, and not on a per-symptom basis. Accordingly, there is some ambiguity in the data from the perspective of illness states and/or symptoms. For example, it is not clear exactly which symptoms (sore throat and/or fever and/or a cough and/or aches, etc.) were reported for a report of an ILI—the ILI reporting code lacks this granularity.

Other approaches are known for identifying and tracking illness state information. For example, illness information may be discussed in forums other than in the context of a report to a healthcare provider. For example, illnesses and/or symptoms may be discussed by individuals on social media platforms—such as Facebook, Twitter, Google Plus, LinkedIn, etc. Sickweather, LLC of Windsor Mill, Md. provides a commercially available data feed that reflects illness data extracted from social media content. Exemplary technology for extracting illness information from social media content is disclosed in U.S. Published Patent Application No. 2014/0108374, the entire disclosure of which is hereby incorporated herein by reference. Such technology further includes associating extracted illness information with a geographical location (such as a latitude/longitude coordinate), and geographical mapping of such information, as discussed therein. An exemplary data feed includes an illness/symptom identifier, latitude and longitude coordinate information, and a timestamp indicating an associated date/time of the social media posting from which the content was extracted.

The data in such data feeds reflect the fine granularity of user-reported illness/condition/symptom that is essentially unrestricted. In other words, a user may place any words in its social media content—flu, sick, congested, “stuffy,” etc. Further, users are more likely to complain in this context of symptoms, rather than illness diagnoses of the type that would be used by healthcare providers. Accordingly, there may be ambiguity in the social mention data, from an illness perspective. For example, when a user complains in a social media posting of a runny nose, it may be unclear whether the complaint relates to an allergy or common cold illness.

These disparate data sources have different purposes, and both have weaknesses in terms of their usefulness to individual consumers, e.g., purchasing appropriate over-the-counter medications for treating observed illnesses/symptoms. What is needed is a system and method for verified reporting of illness/malady/symptom (collectively, “illness”) states in which illness state data is provided to a consumer in a more reliable fashion that is useful to the consumer in purchasing over-the-counter medications for treating observed illnesses/symptoms.

### SUMMARY

The present invention provides a system and method for reporting of illness states in a verified manner, using disparate datasets reflecting illness state reporting. According to one aspect, the present invention provides a verified illness state correlation system (VISCS). The VISCS comprises a processor and a memory operatively connected to the processor, the memory storing executable instructions that, when executed by the processor, cause the system to perform a method for correlating disparate datasets to verify illness reports. The method comprises: receiving a first data set comprising records identifying reported illness events, each record of said first data set comprising aggregated illness event data for each of a plurality of geographic regions; receiving a second data set comprising records identifying reported illness events, each record of said second data set identifying a specific reported illness at a specific geographic location; for each record of said second data set: identifying a matching geographic region of the first data set that encompasses the specific geographic location; determining whether the specific reported illness matches any reported illness event for the matching geographic region; determining a severity level for any matching reported illness state for the matching geographic region; storing the specific reported illness in association with the specific geographic location; and if the severity level exceeds a predetermined threshold, storing, in association with the stored specific reported illness and the stored specific geographic location, a verification flag indicating that the specific reported illness of the second data set is verified by data from the first data set.

According to another aspect, the present invention provides a verified illness state reporting system (VISRS). The VISRS comprises a processor; a display device; and a memory operatively connected to the processor, the memory storing executable instructions that, when executed by the processor, cause the system to perform a method for displaying verified illness reports. The method comprises: receiving input identifying a particular geographical region for which illness reporting shall be displayed via the display device; receiving via a communications network, in response to a request therefor, verified illness state data for the particular geographical region, the verified illness state data comprising a plurality of records, each of the plurality of records identifying a specific reported illness and a corresponding specific geographic location, at least one of the records further including a verification flag indicating verification of the specific reported illness; displaying, via the display device, a map of the particular geographical region, the map including a plurality of markers, each marker corresponding to a respective one of the plurality of records, each marker being displayed in a location corresponding to the respective specific geographic location identified in a corresponding record; and displaying, via the display device, a verification indicator in association with each marker for which the corresponding one of the plurality of records includes a respective verification flag.

According to another aspect, the present invention provides a verified illness state tracking system comprising a VISCS and a VISRS.

Also provided is a computer program product for implementing a method for correlating disparate datasets to verify illness reports, the computer program product comprising a non-transitory computer-readable medium storing executable instructions that, when executed by a processor, cause a computerized system to perform a method for correlating disparate datasets to verify illness reports.

Provided also is a computer program product for implementing a method for displaying verified illness reports, the computer program product comprising a non-transitory computer-readable medium storing executable instructions that, when executed by a processor, cause a computerized system to perform a method for displaying verified illness reports.

### BRIEF DESCRIPTION OF THE FIGURES

An understanding of the following description will be facilitated by reference to the attached drawings, in which:

FIG. 1 is a system diagram showing an exemplary network computing environment in which the present invention may be employed;

FIG. 2A is a schematic diagram of an exemplary special-purpose Verified Illness State Correlation System in accordance with an exemplary embodiment of the present invention;

FIG. 2B is a schematic diagram of an exemplary special-purpose Verified Illness State Reporting System in accordance with an exemplary embodiment of the present invention;

FIG. 3 is a block diagram illustrating relationships among logical components of the exemplary computing device of FIG. 2;

FIG. 4 is a flow diagram illustrating an exemplary method for verification of reported illness states;

FIG. 5 is an image of an exemplary Verified Illness State Reporting System displaying a graphical user interface window displaying a mapping of verified illness state data, and a display of trend data;

FIGS. 6A and 68 are images of an exemplary graphical user interface windows for viewing details of selected verified illness state data;

FIG. 7 is an image of an exemplary Verified Illness State Reporting System displaying a graphical user interface window displaying a user information dashboard;

FIGS. 8A and 8B are graphical user interface windows displaying personalized information content; and

FIG. 9 is a graphical user interface window displaying individual symptoms associated with an exemplary illness state.

### DETAILED DESCRIPTION

The present invention relates to a system and method for reporting of illness states in a verified manner. Generally, the present invention provides a system and method that receives multiple data feeds, collectively including illness report data from disparate data sources, and correlates disparate data sets from the disparate data sources to provide verified reporting of illness states. In a preferred embodiment, the verified reporting is provided in the form of a geographical mapping of reported illness states displayed via a graphical user interface displayed by a computerized device, such that the mapping includes a visual indication of whether a mapped reporting from one data set is verified by being consistent with data from a disparate data set.

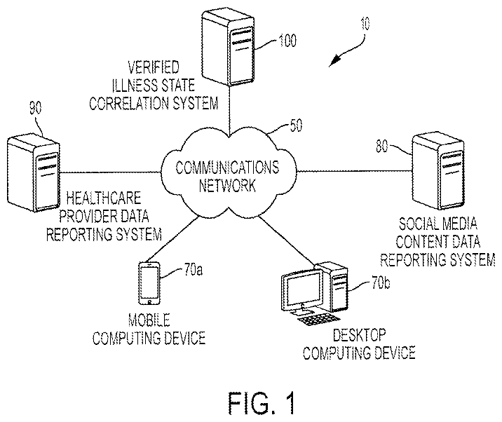

An exemplary embodiment of the present invention is discussed below for illustrative purposes. FIG. 1 is a system diagram showing an exemplary network computing environment in which the present invention may be employed.

The present invention may be understood with reference to the exemplary simplified network environment 10 of FIG. 1. As shown in FIG. 1, the exemplary network environment 10 includes conventional computerized data systems 80, 90. Each of these conventional data reporting systems provides a data feed including illness state report data. Each of these systems provides the data feeds in a conventional manner, e.g., in the form of XML records available in response to API calls. Accordingly, each data reporting system is shown for illustrative an non-limiting purposes as a web server. More particularly, these systems 80, 90 collectively provide disparate data feeds including illness report data sets from disparate data sources. In this example, Healthcare Provider Data Reporting System 90 provides a data feed including data gathered and aggregated from healthcare provider reports, and Social Media Content Data Reporting System 80 provides a data feed including social media content data gathered from social media content. In an alternative embodiments, a single data reporting system may provide the disparate data feeds, and in other alternative embodiments, the data feeds may be provided in different forms.

As further illustrated in FIG. 1, the exemplary network computing environment 10 further includes a Verified Illness State Correlation System (VISCS) 100 in accordance with the present invention. The VISCS 100 receives the data feeds and correlates disparate data sets from the disparate data sources to provide verified reporting of illness states.

As further illustrated in FIG. 1, the exemplary network computing environment 10 further includes Verified Illness State Reporting Systems (VISRSs) 70a, 70b for receiving verified illness state data from the VISCS 100, and displaying a verified report of illness states to a user, in accordance with the present invention.

In this exemplary embodiment, the VISCS 100 is operatively connected to the each VISRS 70a, 70b, and to the Healthcare Provider Data Reporting System 90 and Social Media Content Data Reporting System 80, via a communications network 50, such as the Internet and/or a Virtual Private Network (VPN) connection. Hardware and software for enabling communication of data by such devices via such communications networks are well known in the art and beyond the scope of the present invention, and thus are not discussed in detail herein.

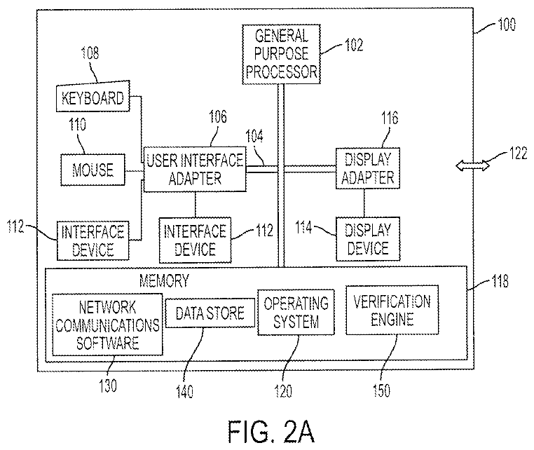

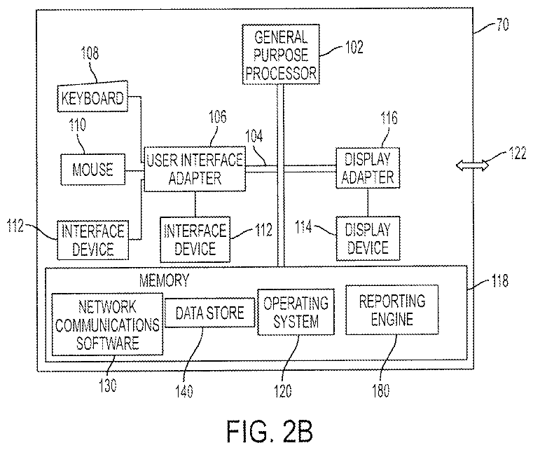

FIG. 2A is a block diagram showing an exemplary Verified Illness State Correlation System (VISCS) 100 in accordance with an exemplary embodiment of the present invention. The VISCS 100 is a special-purpose computer system that includes conventional hardware, e.g. web server hardware, storing and executing both conventional software enabling operation of a general purposes computing system, such as operating system software 120 and network communications software 130, and specially-configured computer software for configuring the general purpose hardware as a special-purpose Verified Illness State Correlation System including a Verification Engine 150 for carrying out at least one method in accordance with the present invention. By way of non-limiting example, the network communications software 130 may include conventional web server software, and the operating system software 120 may include a LAMP stack (Linux, Apache, MySQL, PHP) and implementing Memcached and Varnish, and an API supporting a corresponding app on the VISRS, which may be an iOS app built in a Drupal content management system (CMS).

Accordingly, the exemplary VISCS 100 of FIG. 2A includes a general purpose processor, such as a microprocessor (CPU), 102 and a bus 104 employed to connect and enable communication between the processor 102 and the components of the presentation system in accordance with known techniques. The exemplary presentation system 100 includes a user interface adapter 106, which connects the processor 102 via the bus 104 to one or more interface devices, such as a keyboard 108, mouse 110, and/or other interface devices 112, which can be any user interface device, such as a touch sensitive screen, digitized entry pad, etc. The bus 104 also connects a display device 114, such as an LCD screen or monitor, to the processor 102 via a display adapter 116. The bus 104 also connects the processor 102 to memory 118, which can include a hard drive, diskette drive, tape drive, etc.

The VISCS 100 may communicate with other computers or networks of computers, for example via a communications channel, network card or modem 122. The VISCS 100 may be associated with such other computers in a local area network (LAN) or a wide area network (WAN), and may operate as a server in a client/server arrangement with another computer, etc. Such configurations, as well as the appropriate communications hardware and software, are known in the art.

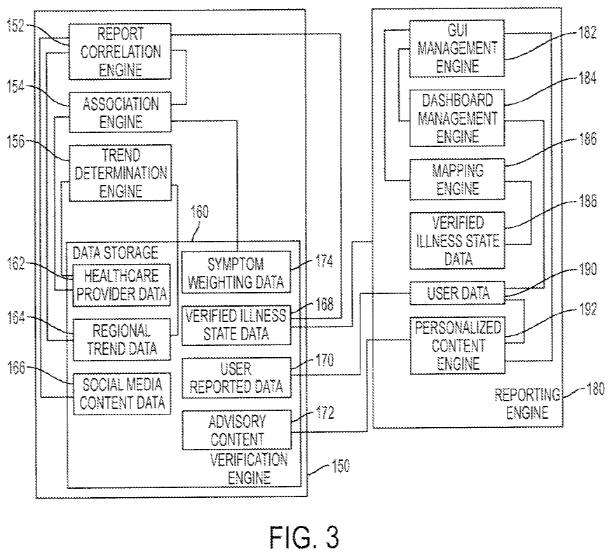

The VISCS 100 is specially-configured in accordance with the present invention. Accordingly, as shown in FIG. 2A, the VISCS 100 includes an illness state correlation application comprising computer-readable, processor-executable instructions stored in the memory for carrying out the methods described herein. Further, the memory stores certain data, e.g. in databases or other data stores shown logically in FIGS. 2A and 3 for illustrative purposes, without regard to any particular embodiment in one or more hardware or software components. For example, FIG. 2A shows schematically storage in the memory 218 (FIG. 3) of Verification Engine software 150 (FIGS. 2 and 3). Optionally, other software and/or data may be stored in a corresponding data store 140 of the memory 118.

The VISRS may include conventional computing device hardware typical of a mobile computing device 70a or desktop computing device (PC) 70b. Any suitable computing devices may be used for the purposes described herein. By way of example, the mobile computing device may be a smartphone, a tablet computer, or the like. Each VISRS 70a, 70b is specially-configured in accordance with the present invention. Each VISRS includes conventional hardware and software and is able to communicate with the VISCS 100, and further includes software (such as a mobile “app”) for configuring the computing device 70a, 70b to communicate with the VISCS 100 and to provide a graphical user interface for displaying a verified report of illness states to a user, in accordance with the present invention.

FIG. 2B is a schematic diagram of an exemplary VISCS 70 (either 70a or 70b). The VISRS may include conventional computing device hardware typical of a mobile computing device 70a or desktop computing device (PC) 70b. Any suitable computing devices may be used for the purposes described herein. By way of example, the mobile computing device may be a smartphone, a tablet computer, or the like. Each VISRS 70a, 70b is specially-configured in accordance with the present invention. Each VISRS includes conventional hardware and software and is able to communicate with the VISCS 100, and further includes software (such as a mobile “app”) for configuring the computing device 70a, 70b to communicate with the VISCS 100 and to provide a graphical user interface for displaying a verified report of illness states to a user, in accordance with the present invention.

Each VISRS 70a, 70b includes components similar to those described above in relation to FIG. 2A, but excludes the Verification Engine 150 shown in FIG. 2A Accordingly, as shown in FIG. 2B, the VISRS 70 includes an illness state reporting application comprising computer-readable, processor-executable instructions stored in the memory for carrying out the methods described herein. Further, the memory stores certain data, e.g. in databases or other data stores shown logically in FIGS. 2B and 3 for illustrative purposes, without regard to any particular embodiment in one or more hardware or software components. For example, FIG. 2B shows schematically storage in the memory 218 of Reporting Engine software 180. Optionally, other software and/or data may be stored in a corresponding data store 140 of the memory 118.

FIG. 3 is a block diagram illustrating selected logical components of the exemplary Verification Engine 150 of FIG. 2A and the Reporting Engine 180 of FIG. 2B. As will be noted from FIG. 3, the Verification Engine 150 includes a data store 160 for storing various data shown logically in FIG. 3. For example, the data store 160 stores Healthcare Provider Data 162. The Healthcare Provider Data is made up of data records, each of which includes data indicating an illness (e.g., illness identifier “ILI”), a regional identifier (e.g., Southern California), and a severity index value (e.g., a number on a scale of 1-12). Byway of example, this information may be provided via a commercially-available data feed communicated to the VISCS 100 via the network 50. In certain embodiments, the records may include a respective severity value for each of a plurality of preceding days. In certain embodiments, the data records may be provided in response to an API call from the VISCS for a specific town/area/postal code within a geographical region, and response will indicate not only the specific town/area/postal code or market region, but also the corresponding data for the corresponding region for which data is available.

The data store 160 further stores Social Media Content Data 166. The Social Media Content Data is made up of data records, each of which includes data indicating a symptom or illness (e.g., “cough” or “runny nose”), a specific location (e.g., street address or latitude/longitude coordinate), and a timestamp (e.g., date/time). This information is, at least in part, extracted form user-generated postings to social media websites/platforms. By way of example, this information may be provided via a commercially-available data feed communicated to the VISCS 100 via the network 50. For example, a commercially-available data feed from Sickweather.com includes a data record indicating a mentioned illness type, and a geographical location, such as latitude and longitude coordinates, indicating a location determined to correspond to the mentioned illness, and a timestamp providing a date/time value. For example, such a record may be provided as XML-formatted content including an illness-identifying code and numerical values indicating latitude and longitude positions within a coordinate system, and a date/time value indicating a time of the mention/social media posting. In certain embodiments, the data records may be provided in response to an API call (e.g., a GET MARKERS call) for recent or geographically-limited records.

The Verification Engine 150 includes a Report Correlation Engine 152 that is responsible for comparing Social Media Content Data 166 with Healthcare Provider Data 162 to determine whether the healthcare provider-derived data corroborates or is otherwise consistent with the social media content-derived data. The Report Correlation Engine 152 may be implemented by suitable software stored in the memory of the VISCS 100. If the provider-reported data corroborates the individual's social mention of an illness/condition/symptom, then in accordance with the present invention the individual's report is considered to be verified, and thus more reliable or persuasive.

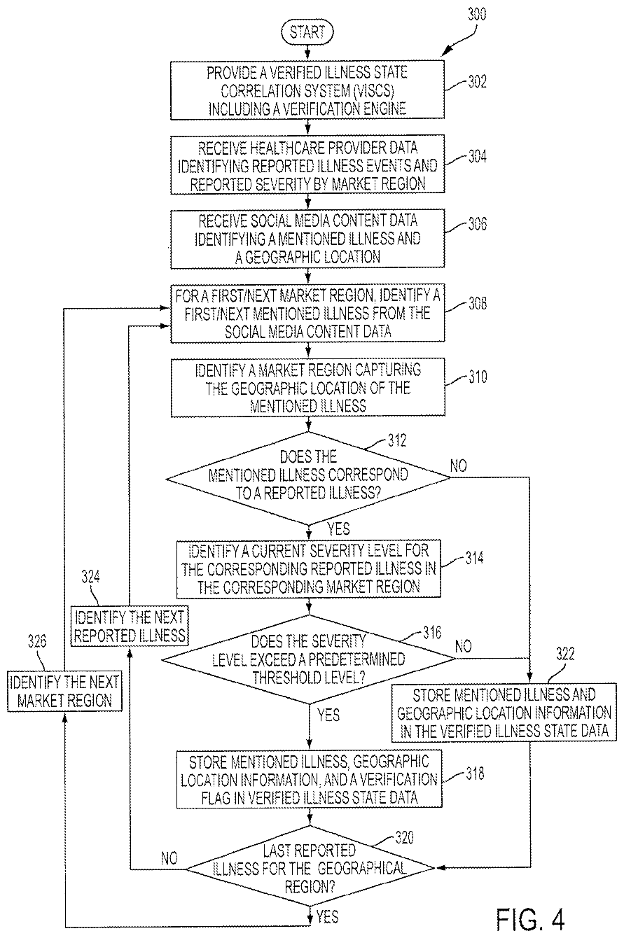

The Report Correlation Engine 152 stores the results of its analysis as Verified Illness State Data 168 in the data store 160. The Verified Illness State Data includes data records, each of which includes an illness state identifier, a location (e.g., latitude and longitude coordinates), and an indication of whether the corresponding illness report has been verified by confirming that the reported illness is consistent with a Healthcare Provider Data. In this example, the indication is a verification flag associated with illness report data. This data (or a geographically-relevant portion of this data) is shared with users' VISRS 70 devices (e.g., smartphones running a corresponding app), so that a geographical mapping of the illness state data may be provided, along with a visual indication of whether each report is verified in that data from one data set is consistent with data from the other. The functioning of the Verification Engine (and its component engines) is discussed below with reference to FIG. 4.

Referring again to FIG. 3, the Verification Engine 150 further includes an Association Engine 154, which may be implemented by suitable software stored in the memory of the VISCS 100. The Association Engine 154 is responsible for associating illness event reports from the Healthcare Provider Data (which may use a first illness taxonomy for reporting purposes), with the illness event reports from the Social Media Content Data (which may use a second illness taxonomy, or no taxonomy, for reporting purposes).

In a simple example, the Association Engine 154 may match a report of ILI from the Healthcare Provider Data (reflecting terminology consistent with an insurance claims processing taxonomy) with a report of “flu” or “influenza” from the Social Media Content Data (reflecting terminology used by a layperson, and further may match both of these to a tracked “FLU” illness state. In this case, the matching is relatively straightforward, since both datasets specifically reference influenza in one form or another.

In a more complex example, the Association Engine 154 includes more complex logic for resolving ambiguous relationships. For example, the Association Engine 154 may process the data records of the Healthcare Provider Data 162 and determine which individual symptoms (e.g., “cough”, “runny nose” etc.) are associated with the various illnesses (e.g., “influenza-like illness”) identified in the Healthcare Provider Data 162. This may be done according to predetermined logic of the Association Engine. Similarly, the Association Engine may associate a Social Media Content Data report of “runny nose” with “ILI” (in the Healthcare Provider Data) and the “FLU” illness state.

In certain embodiments, the VISCS and/or VISRS system provides a graphical user interface permitting an operator to specify weightings for each illness/condition/symptom (hereinafter “symptom”). In such embodiments, the operator-provider symptom weightings are stored as symptom weighting data 174 in the data store 160, and referenced by the Association Engine 154 and are used to allocate the reports, as will be appreciated from FIG. 3. For example, a graphical user interface window may be provided to provide “runny nose” weightings of 30% to the Flu illness state, 30% to the Allergy illness state, and 40% to the Cold illness state, which weightings may be stored in the symptom weighting data 174. In this exemplary embodiment, the Association Engine may treat 30% of the reports of “runny nose” in the Social Media Content Data as reports of “Flu”, so that they may be compared to healthcare provider reported occurrences of ILI, etc. by the Report Correlation Engine 152.

In this exemplary embodiment, the Verification Engine 150 further includes a Trend Determination Engine 156, which may be implemented by suitable software stored in the memory of the VISCS 100. The Trend Determination Engine 156 is responsible for determining illness trends from the Healthcare Provider Data. More specifically, the Trend Determination Engine 156 processes the data records of the Healthcare Provider Data 162 and determines trends, over time, for each illness identifier, for each region. This may be performed in any suitable fashion. In one embodiment, severity index values (e.g., 0-12), for one or more preceding days, are provided in the Healthcare Provider Data. Further, the daily severity index values may be tracked and stored over time for successive days, and may be stored in a memory of the VISCS 100. In such an embodiment, the Trend Determination Engine 156 compares the severity index values over a plurality of days to determine a trend (e.g., rising, falling, or unchanging) for each illness, for each region. The Trend Determination Engine is configured with logic for determining how much of a change will be taken to be an indication of a change in trend. In one example, the Trend Determination Engine compares a preceding day's index value (e.g., 8), or an average of a predetermined number of days' index values (e.g., 8.3) to a current day's index value (e.g., 10) to determine the trend (e.g., rising). The determined trend information is stored by the Trend Determination Engine 156 as Regional Trend Data 164 in the data store 160, as shown in FIG. 3.

FIG. 4 is a flow diagram 300 illustrating an exemplary method for verification of reported illness events. More specifically, the flow diagram 300 illustrates an exemplary method for receiving disparate data sets, and identifying whether individual illness reports from one data set are verified in that they are consistent with reports from a separate data set. Referring now to FIG. 4, the exemplary method begins with providing a VISCS 100 (see FIG. 1) including a Verification Engine 150 (see FIG. 3), as shown at steps 300 and 302. Next, the VISCS 100/Verification Engine 150 receives Healthcare Provider Data, as shown at 304, And Social Media Content Data, as shown at 306. As discussed above, the Healthcare Provider Data identifies reported illness events and reported severity by broad geographical region, in this example, defined market regions. Further, the Social Media Content Data identifies individual-reported illness events mentioned by individuals in social media postings, and specific geographic location data that does not correspond directly to the same location information provided in the Healthcare Provider Data. This data may be stored in the data store 160, as discussed above.

Next, the Verification Engine 150 processes the Social Media Content Data. This is performed iteratively, starting with a first Social Media Content Data record, as shown at 308 in FIG. 4.

Next, the Verification Engine 150 identifies a market region corresponding to the geographic location of the mentioned illness, as shown at 310. This may be performed by the Report Correlation Engine 152. For example, if the first Social Media Content Data record identifies specific latitude and longitude coordinates, these coordinates may be resolved to a particular town (e.g., Fort Washington, Pa.), and the town may be determined to be within one of a predetermined plurality of predefined market regions (e.g., Philadelphia metropolitan area)—namely, one of the market regions that appear in the healthcare provider data. These steps may be performed by the Report Correlation Engine 152.

Next, it is determined if the mentioned illness (from the Social Media Content Data) corresponds to a provider-reported illness (from the Healthcare Provider Data), as shown at 312. This performed by the Association Engine 154. For example, if a Social Media Content Data record indicates a “Flu” illness for a particular location (or a “fever” symptom that is associated with “Flu” by the Association Engine 154), and Healthcare Provider Data includes reports of “Flu” or “ILI”, then it is determined that the mentioned illness corresponds to a reported illness at 312.

If it is determined at 312 that the mentioned illness (e.g. “tired”, or “aches” or “feeling lousy”) does not match a reported illness, then the mentioned illness and the corresponding geographic location information from the Social Media Content Data are stored in the Verified Illness State Data 168, as shown at 322. Notably, this information is stored without an indication of verification/corroboration by the data in another data set. However, this information is stored so that it may be received and displayed, in unverified fashion, on an illness mapping, as discussed further below.

If, however, it is determined at 312 that the mentioned illness does match a reported illness, then the Verification Engine 150 (e.g., the Report Correlation Engine 152) next identifies a current severity level for the corresponding reported illness in the corresponding market region, as shown at 314. For example, the Healthcare Provider Data may indicate a daily severity index value of 10 for “Flu” in the Philadelphia metropolitan market region.

Next, it is determined whether the relevant severity level (e.g., 8) exceeds a predetermined threshold value, as shown at 316. This is performed by the Report Correlation Engine 152 using a threshold value that may be built into the logic of the Report Correlation Engine 152, or may be a user-configurable setting. The threshold value is selected such that a current index value above the threshold reflects prevailing/current conditions such that it is characteristic of the region, and should result in verification of the social mention of illness. For example, if today's severity value (from Healthcare Provider Data) is 10 for the Philadelphia metropolitan market region, and 10 exceeds a predetermined threshold value of 8, then an individual's social mention of flu at a geographic location within the Philadelphia metropolitan market region will result in the social mention report being considered verified by the Healthcare Provider Data.

If the current severity level does not exceed the predetermined threshold, then the mentioned illness and corresponding location data will be stored in the Verified Illness State Data 168, in unverified fashion, as shown at 322. Accordingly, this information is stored so that it may be received and displayed, in unverified fashion, on an illness mapping, as discussed further below.

If, however, the current severity level does exceed the predetermined threshold, then the mentioned illness, corresponding location data and a verification flag will be stored in the Verified Illness State Data 168, as shown at 318. Accordingly, this information is stored so that it may be received and displayed, in verified fashion, on an illness mapping, as discussed further below.

This is repeated for subsequent reported illnesses. Accordingly, the method next involves determining whether the considered reported illness is the last reported illness for the given geographical region, as shown at 320. If so, then the next reported illness is considered, as shown at 324. If not, then the next market region is considered. Either way, flow then returns to 308, where a next mentioned illness is considered, and the process repeats for all mentioned illnesses from the Social Media Content Data. Notably, the entire process of FIG. 4 may be repeated often for each updated set of Social Media Content Data. By way of example, the Social Media Content Data may be updated relatively more frequently, which for example may be updated every 15 minutes, than the Healthcare Provider Data, which may for example be updated only once daily. This continues until all reported illnesses for all market regions have been processed.

In certain embodiments, the Trend Determination Engine 156 determines severity trends as an aggregation of changing severity values in both disparate data sets. By way of example, consider two disparate datasets, namely, a healthcare provider (HCP) data set and a social media content (SMC) data set, each of which reports numerical illness/severity values for each day (or other period) in a particular region. Accordingly, consider that V_{HCP,i} is the reported value for period i, and V_{HCP,j} is the reported value for period j (for the same region). In that case, an average daily trend may be reflected as percentage of change by averaging change vectors for both datasets. By way of example, this may be calculated as follows.

$$V = \frac{( \vec{H C P} ) + ( \vec{S M C} )}{2}$$

$$where$$

$$\vec{H C P} = \frac{( {V}_{HCP , j} - {V}_{HCP , i} ) / ( {V}_{HCP , j} ) \times 100 )}{( j - i )}$$

$$\vec{S M C} = \frac{( {V}_{SMC , k} - {V}_{SMC , 1} ) / ( {V}_{SMC , k} ) \times 100 )}{( k - 1 )}$$

where V is the average percentage change in severity values

HCP is the healthcare provider severity value

SMC is the social media content data severity value

where I, j, k, l and o represent respective time periods (e.g., dates)

where k>I, j>l, and o≠k≠j

In certain other embodiments, the Trend Determination Engine 156 determines trends as a recency-weighted aggregation of severity index values. By way of example, the recency-weighted aggregation may be expressed as percentage of change as weighted by a number of days. The aggregation accounts for disparate trends in disparate data sets, and/or for the asynchronous reporting intervals of the disparate healthcare provider data and social media content data sets. The recency-weighted determination of trends provides greater weight to more recent illness reports, as they are deemed to better reflect current conditions. By way of example, a recency-weighted trend may be calculated as follows:

$$V = \frac{( \vert k - o \vert ) ( \vec{H C P} ) + ( \vert j - o \vert ) ( \vec{S M C} )}{( \vert k - o \vert ) + ( \vert j - o \vert )}$$

$$where$$

$$\vec{H C P} = \frac{( {V}_{HCP , j} - {V}_{H C P , i} ) / ( {V}_{H C P , j} ) \times 100 )}{( j - i )}$$

where V is the percentage change in values of illness values with a recency-weighting providing greater emphasis on recent data/trends

HCP is the healthcare provider severity value

SMC is the social media content data

where I, j, k, l and o represent respective time periods (e.g., dates)

where k>I, j>l, and o≠k≠j

In certain embodiments, the Report Correlation Engine 152 further includes logic for characterizing quantitative index values (stored as Regional Trend Data 164) as qualitative severity measures (e.g., 0-3 is low severity, 3-6 is mild severity, 6-9 is high severity, and 9-12 is extreme severity). These severity characterizations may be stored with the Verified Illness State Data 164 (or elsewhere) for display by the reporting engine 180 as discussed below.

Accordingly, disparate data sets are received by the VISCS 100 and are processed to create the Verified Illness State Data 168 at the VISCS 100. Thus, the illness state correlation application of the VISCS 100 processes disparate data sets to determine correlations between reported illnesses on a location-specific basis to distinguish verified reported illnesses for which there is corroborating information in both data sets, and thus higher relevance, from reported illnesses lacking such corroborating information, and thus having lower relevance.

An individual's VISRS 70 is specially configured with software for displaying at least a relevant portion of the Verified Illness State Data 168. The VISRS 70 includes a Reporting Engine 180 that is responsible for processing received Verified Illness State Data 188 (or at least that portion of it received by the Reporting Engine 180).

The Reporting Engine 180 includes a Graphical User Interface (GUI) Management Engine 182, Dashboard Management Engine 184, Mapping Engine 186 and Personalized Content Engine 192, which may be implemented by suitable software stored in the memory of the VISRS 70 in accordance with the present invention.

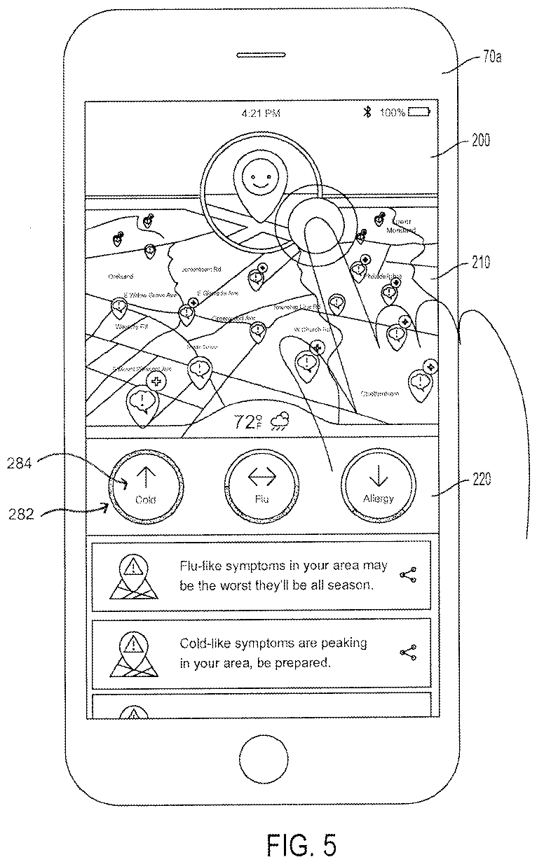

The GUI Management Engine 182 interacts with the remaining engines and is responsible for managing a GUI for displaying information to a user via a display device of the VISRS, and for receiving user input via input devices of the VISRS. User input may be stored as user data 190 in a memory of the VISRS 70. FIGS. 5-9 show exemplary user interface windows 200 created by the GUI Management Engine 182 and displayed via the VISRS 70. More particularly, FIG. 5 shows a graphical user interface window 200 displaying an exemplary mapping 210 of Verified Illness State Data.

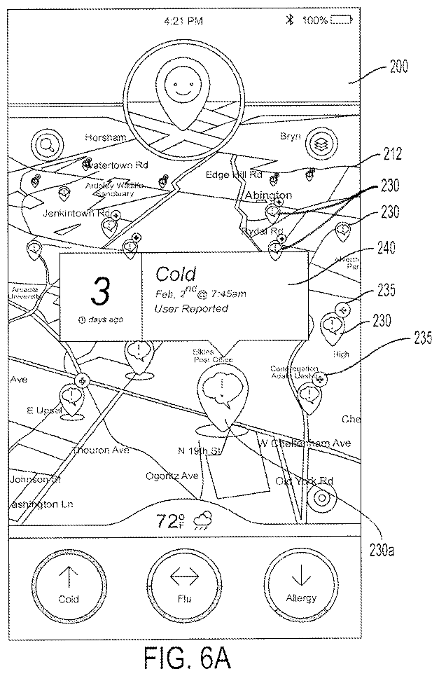

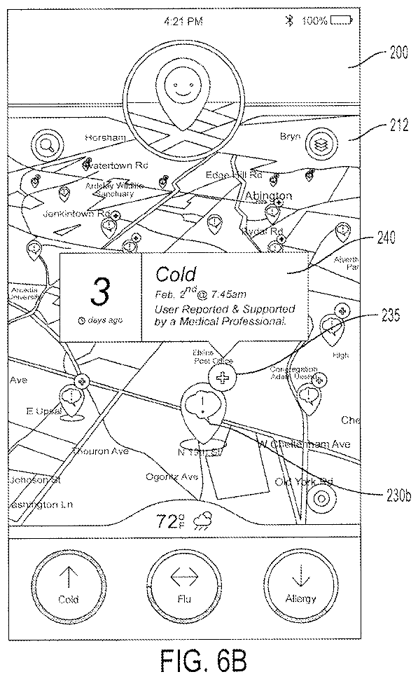

The Mapping Engine 186 determines a current geographical location of the device (e.g., by accessing GPS data available via the device OS), or a selected geographical location (received as user input via the GUI Management Engine 182), and retrieves a relevant portion of Verified Illness State Data 188 corresponding to that location, which is stored in memory of the VISRS 70. For example, if the Mapping Engine determines that the relevant portion includes data for the Philadelphia metropolitan region, the Mapping Engine 186 will retrieve that portion. The Mapping Engine 186 is responsible for creation of an image displaying a mapping 210 of the relevant portion of the Verified Illness State Data 188, as shown in FIGS. 5-6B. To do so, the Mapping Engine may communicate via the network, e.g., via an appropriate API, to cause creation of the mapping. By way of non-limiting example, for an Apple iOS app, the Mapping Engine may just fetch markers and clusters via a corresponding server API, and display those via Apple's MKMapView object, e.g., by passing the geo-coordinates of the top right and bottom left of the map.

The Mapping Engine 186 creates a mapping that includes not only markers positioned in a location-appropriate images on a map, but also includes a visual indicator indicating whether the illness report corresponding to an associated marker has been verified, by consistency with a second data set. Accordingly, a user viewing the mapping can tell which illness reports are verified, and which are not, by browsing the map. For example, FIGS. 6A and 6B show graphical user interface windows 200 displaying a map 212 showing markers 230 representing illness reports. The marker locations correspond to the locations of social media mentioned illness reports identified in the Social Media Content Data. Further, the Figures show verification indicators 235 for those reported illnesses that are verified, in that the reported illness is consistent with healthcare provider reported data from the Healthcare Provider Data. FIG. 6A shows that by selecting (e.g., touching in a touch-based interface) a selected marker 230a, associated illness data is displayed in a detail panel 240. In this case, the marker 230a lacks a verification indicator 235, and the panel 240 notes that the associated data is user-reported only—from the Social Media Content Data. In contrast, FIG. 6B shows that by selecting another marker 230b, associated illness data is again displayed in a detail panel 240. In this case, however, the marker 230b includes a verification indicator 235, and the associated panel notes that the associated data is both user-reported (from the Social Media Content Data), and that the report is consistent with data from a second source—namely, the Healthcare Provider Data. Accordingly, the corresponding illness report is deemed to be verified, and thus more trustworthy.

Accordingly, the reported illnesses are indicated by markers displayed on the map of the particular geographical region of interest via the display device of the computerized system, and reported illnesses supported by corroborating information in the disparate data sets are indicated by verification indicators displayed on the map in association with such markers. Thus, reported illnesses are processed programmatically by the illness state reporting application and are displayed on a geographically-relevant basis in differentiated fashion to provide a visual indication of verified and unverified reported illnesses via the display device of the computerized system when the illness state reporting application is active on the computerized system.

In certain embodiments, the markers are coded—e.g., by color and/or shape, to convey a severity level associated with the healthcare provider data. For example, data indicating a severity level of 0-3 may be shown with a blue marker, and a severity level of 9-12 may be shown with a red marker.

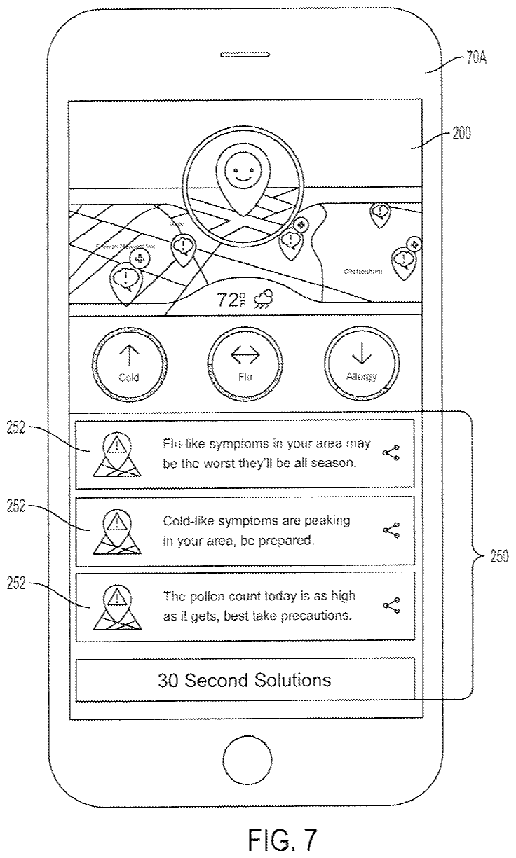

The Dashboard Management Engine 184 receives user data and communicates with the GUI Management Engine 182 to cause display of customized alerts. The alerts are customized for the relevant geographical region being displayed in the graphical user interface window. If the geographical region being displayed includes more than one market region, severity indices and/or trends may be aggregated (e.g., by averaging values/trends for each market) for the various market regions shown. FIG. 7 shows a graphical user interface window 200 displaying a user information dashboard 250 displaying customized alerts 252. The alerts indicate trend information with respect to reported illness states in the relevant geographical area. The user can use these alerts in seeking treatment for any observed symptoms/illness states, for purchasing appropriate over-the-counter medications, etc. The alerts are selectively displayed in the graphical user interface window for each illness state (e.g., Cold, Flu, Allergy) as a function of a current severity index value and a predetermined threshold value. For example, if the predetermined threshold value is 10 for cold and 9 for allergy, the system will display cold-related alerts to the user when the user's device is in a geographical location within a market region for which the current severity index value is 10 or above, and will display allergy-related alerts to the user when the user's device is in a geographical location within a market region for which the current severity index value is 9 or above.

The Dashboard Management Engine 184 is further responsible for communicating with the GUI Management Engine 182 to cause display of infographics in a dashboard panel 220. Each exemplary infographic 280 includes a current severity indicator. In the example of FIG. 5, the current severity indicator 282 is represented by a circular bar graph. The severity indicator indicates in graphical form the current severity index value (e.g., 8) relative to the scale (e.g., 0-12) for each illness state (e.g., Cold, Flu, Allergy). Accordingly, the current severity indicator reflects the calculated severity levels determined by the Report Correlation Engine 152 (e.g., low, mild, high, extreme), and provides at a glance a visual indication of relative severity. These are displayed for the current location of the user's device or a selected map region, and may be color-coded to indicate severity.

Further, the Dashboard Management Engine 184 is responsible for displaying a trend indicator in the dashboard panel 220. In the example of FIG. 5, the trend indicator 284 is represented by an arrow. The trend indicator indicates in graphical form the current severity trend, over time, for each illness state. Accordingly, the trend indicator reflects the calculated trend determined by the Trend Determination Engine 156 (e.g., rising, falling, unchanging), and provides at a glance a visual indication of a change in severity over time.

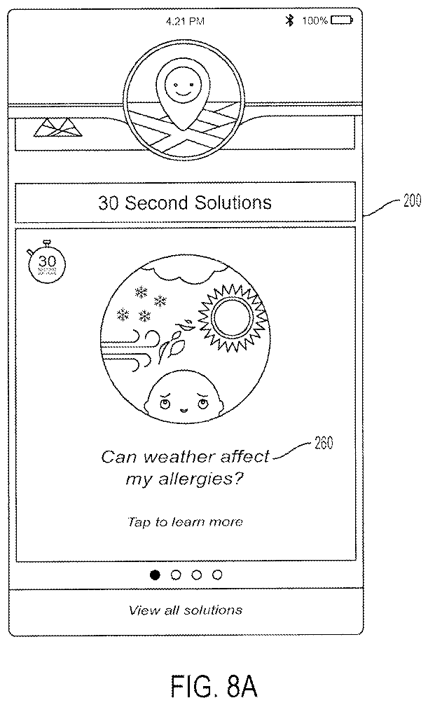

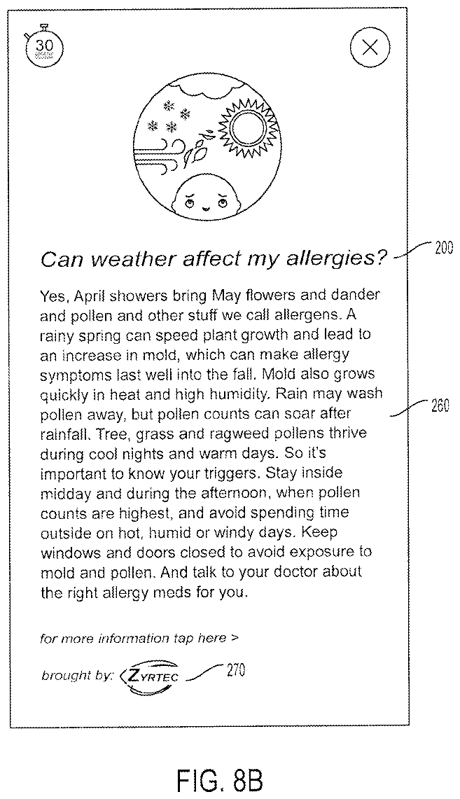

The Personalized Content Engine 192 receives user data and communicates with the GUI Management Engine 182 to cause display of relevant information content 260. For example, if the user is concerned about allergies, the user may choose to receive a display of personalized information content including allergy-related information. FIGS. 8A and 8B are exemplary graphical user interface windows displaying personalized information content relating to allergies. Optionally, an advertisement and/or a hyperlink 270 is provided as a path to purchase a relevant product, such as an OTC medication, for treating the related illness (here, allergies). The Personalized Content Engine 192 may determine whether information content is relevant as a function of geographically-relevant illness trends, individual illness reports, user input, etc. The Personalized Content Engine 192 may selectively retrieve relevant information content (as determined by user input/data) from a repository of advisory content 172 stored in the data store 160 of the VISCS 100, as will be appreciated from FIG. 3.

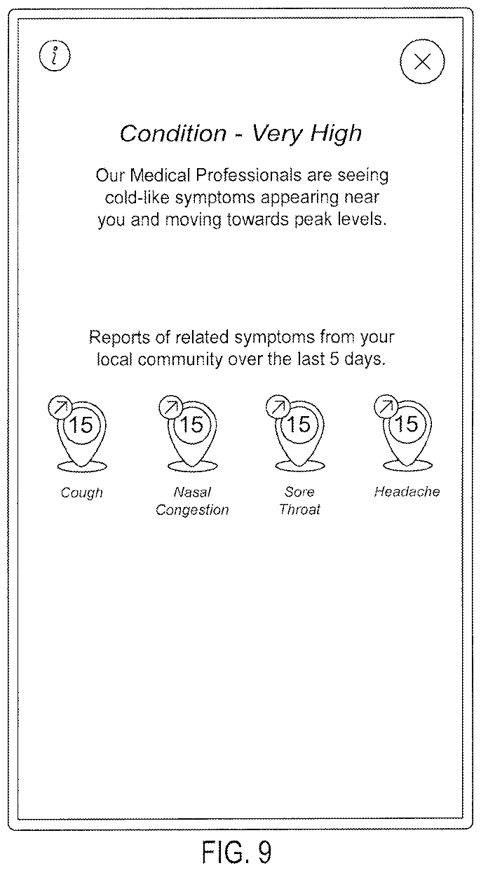

FIG. 9 is a graphical user interface window displaying individual symptoms associated with the illness states. The individual symptoms reflect symptoms from the social media content data or healthcare provider data that are associated with a selected illness state. This type of breakdown may be provided when the data feeds provide such information. For example, if cough, nasal congestion, sore throat, and headache symptoms are associated with a Cold illness state, this user interface window may show that this association. In this case, a symptom report count (e.g., 15 instances in this example, for the relevant geographical region being displayed/considered) is shown in conjunction with each symptom, as well as a trend indicator (e.g., rising arrow in this example) associated with the corresponding illness state (e.g., Cold illness state in this example). In certain instances, symptoms may be displayed in conjunction with an advertisement and/or a hyperlink or other path to purchase to products for treating (or being otherwise associated with) the symptoms, or may be displayed in conjunction with information content related to the symptoms.

It will be noted from FIG. 3 that in certain embodiments, user data 190 received at the VISRS 70 is communicated to the GUI Management Engine 182, which can immediately add corresponding markers to the map in cooperation with the Mapping Engine 186 and the GUI Management Engine 182. See FIG. 5. In certain embodiments, that user data is communicated back to the VISCS 100 and stored as user reported data 170, which may be used for verification purposes, and ultimately may be added (verified or not) to the Verified Illness State Data 168, as will be appreciated from FIG. 3.

It is noted that the exemplary embodiment(s) discussed above relate to a limited context of Cold, Flu and Allergy illness states. However, it should be appreciated that the present invention may be used for a variety of other states/illness states, and in a variety of other contexts. By way of example, the present invention can be applied in the contexts of sun/skin care, oral care, pediatric care, etc.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

Mile there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

1-12. (canceled)

13. A verified illness state reporting system comprising:
a processor;
a display device; and
a memory operatively connected to the processor, said memory storing an illness state reporting application comprising executable instructions that, when executed by the processor, cause the system to perform a method for displaying verified illness reports via the display device, the method comprising:
receiving input identifying a particular geographical region for which illness reporting shall be displayed via the display device under control of the illness state reporting application;
receiving via a communications network, in response to a request therefor, verified illness state data for the particular geographical region, the verified illness state data comprising a plurality of records, each of the plurality of records identifying a specific reported illness and a corresponding specific geographic location, at least one of the records further including a verification flag indicating verification of the specific reported illness;
displaying, via the display device, a map of the particular geographical region, the map including a plurality of markers, each marker corresponding to a respective one of the plurality of records, each marker being displayed on the display device in a location corresponding to the respective specific geographic location identified in a corresponding record; and
displaying, via the display device, a verification indicator in association with each marker for which the corresponding one of the plurality of records includes a respective verification flag.

14-45. (canceled)
